(19) United States
(12) Patent Application Publication
(10) Pub. No.: US 2009/0298822 A1
(43) Pub. Date: Dec. 3, 2009

(54) Use of Activators of Soluble Guanylate Cyclase for Treating Reperfusion Damage

(75) Inventors: Thomas Krahn, Hagen (DE); Johannes-peter Stasch, Solingen (DE); Gerrit Weimann, Koln (DE); Wolfgang Thielemann, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Health Care, Leverkusen (DE)

(21) Appl. No.: 11/922,838

(22) PCT Filed: Jul. 6, 2006
(86) PCT No.: PCT/EP2006/006600

§ 371 Date: Aug. 3, 2009

(30) Foreign Application Priority Data
Jul. 6, 2005 (DE) 10 2005 031 576.3

(51) Int. Cl. A61K 31/5377; A61K 31/197; A61K 31/506; A61P 7/00

(52) U.S. Cl. 514/234.2; 514/567; 514/256; 514/231.5; 514/236.8

(57) ABSTRACT

The present invention relates to the use of compounds for manufacturing a pharmaceutical product/medicament for the prophylaxis and/or treatment of reperfusion damage.

The present invention relates to the use of compounds for manufacturing a pharmaceutical product/medicament for the prophylaxis and/or treatment of reperfusion damage.

Reperfusion damage generally occurs after the end of a prolonged ischemic period, e.g. as a consequence of toxic metabolites which invade and accumulate after restoration of the blood flow and/or of the massive release of calcium ions in excitable cells. This damage frequently occurs after vascular occlusions, specifically after acute arterial occlusions, when a compensating collateral circulation is lacking (so-called infarctions). The best-known forms are myocardial infarction and cerebral infarction (stroke). Whereas early restoration of the blood flow by a thrombolysis after a temporary ischemia can prevent or reduce the extent of cell damage (infarct size), the reperfusion may nevertheless cause to a certain extent dysfunctions for example of the heart, or cell death. It is therefore of great clinical value to find medicaments which maintain the normal function for example of the heart during reperfusion and during the various types of heart surgery.

It is known that ischemic reperfusion damage and cellular damage associated therewith occur for example in association with: myocardial infarction, replacement of coronary arterial vessels, especially open-thorax heart surgery, angina, peripheral vascular occlusive diseases, stroke, tissue and organ transplants (e.g. heart, liver, kidney, lung), general surgery, acute renal failure and hypoperfusion of organs (e.g. lung, heart, liver, bowel, pancreas, kidney, extremities or brain).

It is known that mechanisms (e.g. NO-releasing substances) which lead to an increase in the intracellular messenger cGMP may also lead to a reduction in reperfusion damage if the treatment with these substances is started before or, in some cases, during the ischemic period. The use before an ischemic period is generally known as prophylaxis/protection and/or preconditioning and includes cellular protection, specifically the protection of excitable cells (e.g. nerve and muscle cells). Treatment after an ischemic period is correspondingly referred to as postconditioning.

Elevated cGMP levels may lead to protection of cells, tissues and organs from reperfusion damage. The activation (agonists) of soluble guanylate cyclase leads to an increase in the intracellular messenger cGMP. It has surprisingly now been found that the compounds of the invention activators of soluble guanylate cyclase (compounds of formulae I to IV) are especially suitable for the manufacture of pharmaceutical substances/medicaments for the prophylaxis and/or treatment and the limiting of reperfusion damage in mammals, especially in humans.

Compound (I) corresponds to the following formula:

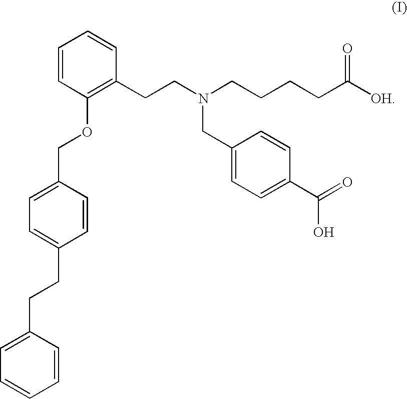

Compound (I), the preparation and use thereof as pharmaceutical have been disclosed in WO 01/19780.

Compound (II) corresponds to the following formula:

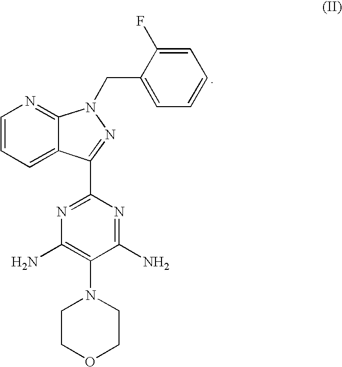

Compound (II), the preparation and use thereof as pharmaceutical have been disclosed in WO 00/06569.

Compound (III) corresponds to the following formula:

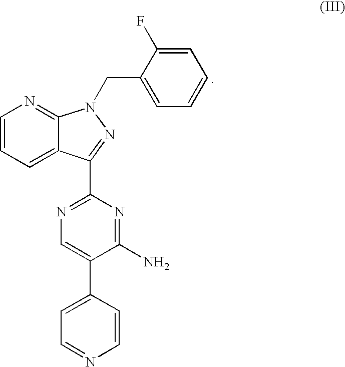

Compound (III), the preparation and use thereof as pharmaceutical have been disclosed in WO 00/06569 and WO 02/42301.

Compound (IV) corresponds to the following formula:

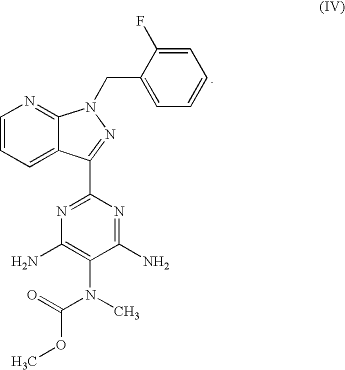

Compound (IV), the preparation and use thereof as pharmaceutical have been disclosed in WO 00/06569 and WO 03/095451.

Compound (IVa) corresponds to the following formula:

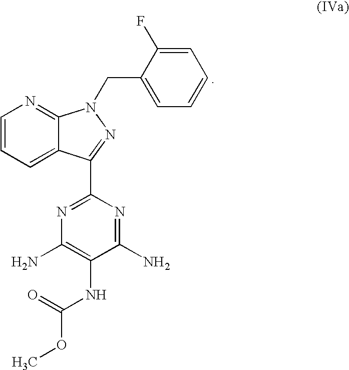

Compound (IVa), the preparation and use thereof as pharmaceutical have been disclosed in WO 00/06569 and WO 03/095451.

Compound (V) corresponds to the following formula:

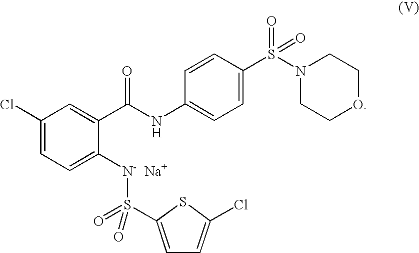

Compound (VI) corresponds to the following formula:

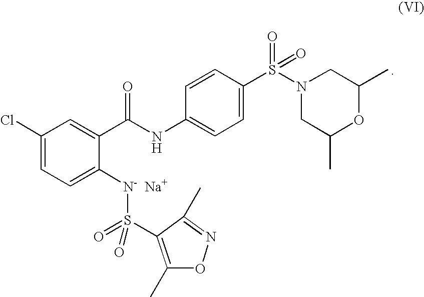

Compounds (V) and (VI), the preparation and use thereof as pharmaceutical have been disclosed in WO 00/02851.

The present invention relates to the use of compounds of the formulae (I-VI) and the salts, hydrates, hydrates of the salts thereof for the manufacture of a medicament for the treatment of reperfusion damage.

An additional exemplary embodiment of the present invention includes the procedure for the prophylaxis and/or treatment of reperfusion damage by using at least one of the compounds of the formulae (I-VI).

The present invention further relates to pharmaceuticals comprising at least one compound of the invention and at least one or more further active ingredients, especially for the treatment and/or prophylaxis of the aforementioned disorders.

The compounds of the invention may have systemic and/or local effects. They can for this purpose be administered in a suitable way, such as, for example, by the oral, parenteral, pulmonary, nasal, sublingual, lingual, buccal, rectal, dermal, transdermal, conjunctival or otic route or as implant or stent.

The compounds of the invention can be administered in suitable administration forms for these administration routes.

Administration forms suitable for oral administration are those which function according to the state of the art and deliver the compounds of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, injection and infusion preparations in the form of solutions, suspensions, emulsions, lyophilizates or sterile powders.

Examples suitable for other administration routes are medicinal forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and masking tastes and/or odors.

The present invention further relates to medicaments which comprise at least one compound of the invention, normally together with one or more inert, non-toxic, pharmaceutically suitable excipients, and to the use thereof for the aforementioned purposes.

It has generally proved advantageous to administer amounts of about 0.01 to 5000 mg/kg, preferably about 0.5 to 1000 mg/kg, of body weight per day to achieve effective results.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The formulations can moreover comprise, appropriate for the intervention, active substance between 0.1 and 99% active ingredient, in a suitable manner 25-95% in the case of tablets and capsules and 1-50% in the case of liquid formulations, i.e. the active ingredient should be present in amounts sufficient to achieve the stated dose range.

An additional exemplary embodiment of the present invention is the use of a combination of one or more of the compounds of the invention with one or more other substances. Suitable combinations of substances are for example substances which are used for the prophylaxis and/or treatment of infarctions and reperfusion damage. In this connection, by way of example and preferably are cGMP-elevating substances such as NO-releasing substances, inhibitors of phosphodiesterases, thrombolytics and adenosine agonists.

### EXPERIMENTAL SECTION

### Reduction of the Infarct Size and Further Reperfusion Damage on the Isolated Heart by Administering an NO-Independent Activator of Soluble Guanylate Cyclase

Determination of the infarct size and the procedure for the experiment follows the method described by Zhang et al. in J. Cardiovasc. Pharmacol., 42, 764-771, 2003.

Rabbits of both sexes of the white New Zealand breed (2-3 kg body weight) were anesthetized with sodium pentbarbital (30 mg/kg i.v.) and ventilated. Following a surgical procedure, the isolated heart was rapidly transferred into a Langendorff setup. The isolated heart is in this case fixed at the aortic root and subjected to retrograde perfusion with a Krebs buffer consisting of (in mM): NaCl 118.5; KCl 4.7; MgSO₄ 1.2; KH₂PO₄ 1.2; NaHCO₃ 24.8; CaCl₂ 2.5 and glucose 10. The buffer is gassed with a mixture of 95% O₂ and 5% CO₂ at a pH of 7.35-7.45 and a temperature of 38° C. All hearts were able to equilibrate for at least 30 minutes before the start of the test protocol.

The infarct size was determined at the end of the experiment by rapidly removing the isolated heart from the Langendorff setup. After a washing step in physiological saline, the coronary artery was closed again and fluorescent microspheres were infused into the heart in order to demonstrate the risk zone or the ischemic area as non-fluorescent tissue. After the heart had been weighed and deep-frozen, it could be cut into slices 2 mm thick. These slices were incubated in 1% triphenyltetrazolium chloride (TTC) in sodium phosphate buffer at 37° C. for 20 minutes. The viable tissue is stained dark red during this, whereas the necrotic tissue is not stained and appears brownish.

All hearts (in each case n=6 per group) were subjected to a 30-minute ischemia by coronary ligature and a 120-minute reperfusion phase. Control hearts were subjected only to an ischemia and reperfusion. In the treatment group, the hearts were perfused with the NO-independent activator of soluble guanylate cyclase. The conclusion can be summarized as the fact that activators of soluble guanylate cyclase are suitable for reducing the infarct size and diminishing reperfusion damage.

1. A method for the prophylaxis and/or treatment of reperfusion damage comprising administering a medicament comprising an effective amount of a compound of the formulae (I-VI) 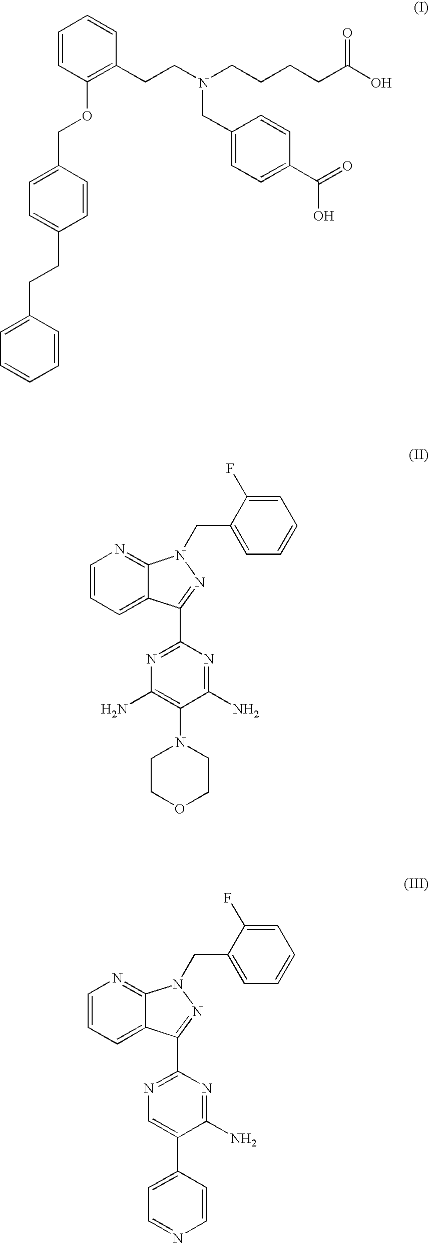 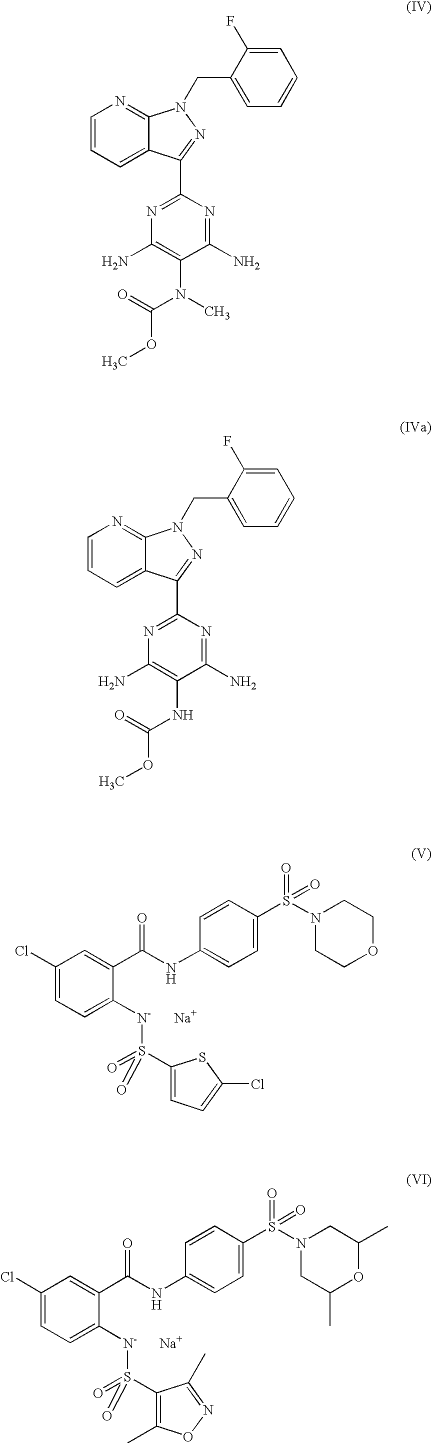
or a salt, hydrate, or hydrate of a salt thereof.

2. The method as claimed in claim 1, in which the medicament is used for an oral dosage form.

3. The method as claimed in claim 1, in which the medicament is given intravenously.

4. The method as claimed in claim 1, in which the medicament is preventive.

5. The method as claimed in claim 1, in which the medicament is used for the prophylaxis and/or treatment of reperfusion damage.

6. A pharmaceutical composition for the treatment of reperfusion damage, which comprises at least one substance as described in a claim 1.

7. The pharmaceutical composition as claimed in claim 6, which additionally comprises a medicament selected from the group of inhibitors of phosphodiesterases, thrombolytics and adenosine agonists.
